(19) United States
(12) Patent Application Publication
(10) Pub. No.: US 2011/0282069 A1
(43) Pub. Date: Nov. 17, 2011

(54) HIGH-PURITY FEBUXOSTAT AND THE METHOD FOR PREPARATION

(75) Inventors: Xingguo Zhou, Chongqing (CN); Xuemin Tang, Chongqing (CN); Ruina Shi, Chongqing (CN); Wenrun Ye, Chongqing (CN); Jie Luo, Chongqing (CN); Jie Deng, Chongqing (CN); Bin Fan, Chongqing (CN)

(73) Assignee: CHONGQING PHARMACEUTICAL RESEARCH INSTITUTE CO/. LTD., Chongqing (CN)

(21) Appl. No.: 13/145,499

(22) PCT Filed: Jan. 20, 2010
(86) PCT No.: PCT/CN10/70271

§ 371 Date: Jul. 20, 2011

(30) Foreign Application Priority Data
Jan. 20, 2009 (CN) 200910103095.9

(51) Int. Cl. C07D 277/56

(52) U.S. Cl. 548/201

(57) ABSTRACT

A febuxostat which purity is not less than 99.0%, method for preparation thereof, and pharmaceutical composition thereof. The method for preparation includes recrystallizing febuxostat in a mixed solvent. The said pharmaceutical composition can be used in manufacture of medicaments for treating diseases associated with hyperuricemia.

### FIELD OF THE INVENTION

The present invention belongs to the field of medicinal chemistry. Specifically, the present invention relates to a high-purity compound comprising 2-[3-cyano-4-(2-methylpropoxy)phenyl)-4-methyl-5-thiazolecarboxylic acid (febuxostat), and a method for preparing the high-purity febuxostat. The present invention further relates to a composition comprising the high-purity febuxostat and optional pharmaceutically acceptable carriers and excipients, and a use of the high-purity febuxostat in the preparation of a pharmaceutical composition.

### BACKGROUND OF THE INVENTION

Febuxostat (chemical name: 2-[3-cyano-4-(2-methylpropoxy)phenyl)-4-methyl-5-thiazolecarboxylic acid) has the following chemical structure:

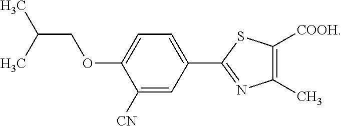

Febuxostat is the first novel non-purine type xanthine oxidase inhibitor, which can be used for effective and safe treatment of diseases associated with high level of uric acid, such as gout. Febuxostat has already been approved for marketing in EU, and has been submitted with FDA for New Drug Registration and obtained the recommendation for approval.

Patent Publication WO92/09279 is a compound patent application of this drug, which relates to 110 structural analogues of febuxostat and methods for their synthesis and purification, as well as their indications and so on. Patent JP10-45733 discloses two other processes for synthesizing febuxostat. JP6-345724 discloses a method for synthesizing febuxostat using a double-cyano-substituted benzene compound. These patent documents provide a variety of methods for synthesizing febuxostat, and methods for synthesizing structural analogues thereof. The synthesis process provided by patent JP10-45733 is relatively suitable for industrialization due to its shorter reaction scheme, readily available raw materials (not involving controlled toxic materials), mild reaction conditions, high yield of process and less special wastes. In the above processes in the art for preparing febuxostat, the last step always involves the hydrolysis of ester intermediates, whereas the hydrolysis step usually introduces some impurities into the product febuxostat since the ester intermediates also contain other hydrolyzable groups such as cyano group in their structure. In addition, the hydrolysis of some impurities entrapped in the ester intermediates may also produce some impurities in the product febuxostat. Hence, a variety of impurities usually are present in the product febuxostat. However, the studying of impurities in febuxostat is rarely found in the prior art. The presence of impurities may bring about potential risks on drug safety, for example, may be associated with some adverse reactions; and may even affect the long-term stability of drugs.

### SUMMARY OF THE INVENTION

In order to further improve the quality of febuxostat and effectively control the impurities contained therein, the inventors of the present invention prepared febuxostat using the method of patent JP10-45733 and studied the impurities contained therein. The inventors found in the study that three impurities with relatively high levels were present in the final product febuxostat, and these impurities were not readily to be reduced or removed by the purification methods in the prior art, such as recrystallization using a single solvent such as methanol, ethanol, acetone and so on.

Therefore, aiming at the problems found in the study, the inventors prepared and purified the three major impurities, determined their structures, and provided a new purification method for refining febuxostat using a mixed solvent to effectively reduce or remove the three impurities. By the purification method, a high-purity febuxostat was obtained. The high-purity febuxostat can also be used for the preparation of a pharmaceutical preparation to reduce the initial content of impurities in the preparation, which is favorable to the storage and safety improvement of the preparation.

Therefore, the present invention first provides a high-purity febuxostat with a content not less than 99.0%. In one aspect, the high-purity febuxostat is obtained by recrystallizing febuxostat in a mixed solvent comprising two or more solvents. In a preferred aspect, the solvents are selected from alcohols, heterocyclic cycles, ketones, esters, ethers, halogenated alkanes, especially from alcohols, ketones, esters or heterocyclic cycles. In a more preferred aspect, the alcohols are selected from methanol, ethanol, propanol, isopropanol, n-butanol, isobutanol; the ketones are selected from acetone and cyclohexanone; the esters are selected from ethyl acetate and methyl acetate; the heterocyclic cycles are selected from tetrahydrofuran, dioxane and thiazole. In a particularly preferred aspect, the solvent is a mixed solvent of an alcohol and a heterocyclic cycle in a volume ratio of 10:1 to 1:10, preferably 10:2 to 10:5. In a more particularly preferred aspect, the solvent is a mixed solvent of methanol and tetrahydrofuran in a volume ratio of 5:1.

The invention also provides a method for preparing the above high-purity febuxostat, the method comprises recrystallizing febuxostat in a mixed solvent comprising two or more solvents. In the method of the present invention, the solvent is preferably a mixed solvent consisted of two or more solvents selected of the group consisting of alcohols, heterocyclic cycles, ketones, esters, ethers, halogenated alkanes, among which the more preferred solvents are alcohols, ketones, esters or heterocyclic cycles. Wherein, preferably, the alcohols are selected from methanol, ethanol, propanol, isopropanol, n-butanol and isobutanol; the ketones are selected from acetone and cyclohexanone; the esters are selected from ethyl acetate and methyl acetate; the heterocyclic cycles are selected from tetrahydrofuran, dioxane and thiazole. In a particularly preferred aspect, the solvent is a mixed solvent of an alcohol and a heterocyclic cycle in a volume ratio of 10:1 to 1:10, preferably 10:2 to 10:5. In a more particularly preferred aspect, the solvent is a mixed solvent of methanol and tetrahydrofuran in a volume ratio of 5:1.

On another hand, the inventors further prepared, purified and identified the three main impurities in the febuxostat obtained by the method of the prior art: 2-[3-carbamyl-4-(2-methylpropoxy)phenyl]-4-methyl-5-thiazole acid (FBZA), 2-[3-carboxy-4-(2-methylpropoxy)phenyl]-4-methyl-5-thiazole acid (FBZB) and 2-[3-formyl-4-(2-methylpropoxy)phenyl]-4-methyl-5-thiazole acid (FBZC), and their structures are shown as follows:

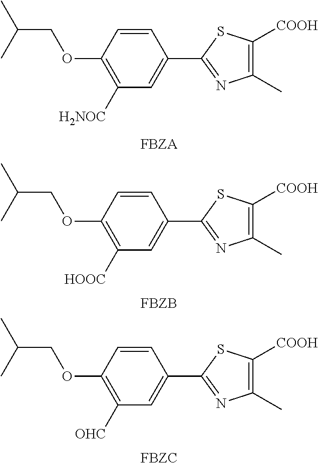

Preferably, the high-purity febuxostat of the present invention is of a content of not less than 99.0%, and the impurity FBZA contained therein is not higher than 0.5% and the impurity FBZB contained therein is not more than 0.25%.

In another aspect, the present invention further provides a pharmaceutical composition, comprising the high-purity febuxostat of the present invention and optional pharmaceutically acceptable carriers and excipients. In addition, the present invention further provides a use of the high-purity febuxostat of the present invention in manufacture of a pharmaceutical composition, especially in manufacture of a pharmaceutical composition for preventing and treating diseases associated with high level of uric acid (for example gout).

In further another aspect, the present invention provides a use of FBZA and/or FBZB as a standard reference for quality control of the high-purity febuxostat of the present invention. Similarly, the present invention also provides a use of FBZC as a standard reference for quality control of the high-purity febuxostat of the present invention. The standard reference for quality control can be used in, for example, the assay of febuxostat and the determination of related substances, for example, as an internal standard or external standard reference substance of high-performance liquid chromatography (HPLC), alternatively, their correlation factors can be measured first and then used together with their retention time relative to that of febuxostat in the HPLC to perform the determination.

In addition, the present invention further provides a method for treatment of a disease, including gout, associated with high level of uric acid, comprising administering a patient in need of the treatment with the high-purity febuxostat of the present invention.

### DETAILED DESCRIPTION OF THE INVENTION

In the first aspect of the present invention a high-purity febuxostat is provided.

To achieve this purpose, the present invention provides a high-purity febuxostat, in which the content of febuxostat is not less than 99.0%, preferably not less than 99.5%, 99.6%, 99.7%, 99.8%, or 99.9%.

In one aspect, the high-purity febuxostat of the present invention is obtained by recrystallizing febuxostat in a mixed solvent comprising two or more solvents. Wherein, the raw material febuxostat used in the preparation of the high-purity febuxostat is a febuxostat with normal purity, which can be prepared by any possible means, and the content thereof is generally lower than 99%, for example, lower than 99.0%, lower than 98.5%, lower than 98.0%, lower than 97.5%, lower than 97.0%, lower than 96.0%, lower than 95.0%, even lower than 90.0%, and the like. In a preferred aspect, the solvent is selected from alcohols, heterocyclic cycles, ketones, esters, ethers, halogenated alkanes; especially from alcohols, ketones, esters or heterocyclic cycles. In a more preferred aspect, the alcohols are selected from methanol, ethanol, propanol, isopropanol, n-butanol, isobutanol; the ketones are selected from acetone and cyclohexanone; the esters are selected from ethyl acetate and methyl acetate; the heterocyclic cycles are selected from tetrahydrofuran, dioxane and thiazole. In particular, the solvent is a mixed solvent of an alcohol and a heterocyclic cycle in a volume ratio of 10:1 to 1:10, preferably 10:2 to 10:5. More specifically, the solvent is a mixed solvent of methanol and tetrahydrofuran in a volume ratio of 5:1.

The invention further provides a high-purity febuxostat, wherein the content of febuxostat is not less than 99.0%, and the content of 2-[3-carbamyl-4-(2-methylpropoxy)phenyl]-4-methyl-5-thiazole acid (FBZA) as impurity therein is not more than 0.5%, 0.3%, 0.2%, 0.15%, 0.1%, or 0.05%, and the content of 2-[3-carboxy-4-(2-methylpropoxy)phenyl]-4-methyl-5-thiazole acid (FBZB) as impurity therein is not more than 0.25%, 0.2%, 0.15%, 0.1%, or 0.05%.

In another particular embodiment of the present invention, a high-purity febuxostat is provided, wherein the content of febuxostat is not less than 99.5%, and the content of 2-[3-carbamyl-4-(2-methylpropoxy)phenyl]-4-methyl-5-thiazole acid (FBZA) as impurity therein is not more than 0.5%, 0.4%, 0.3%, 0.2%, 0.15%, 0.1% or 0.05%, and the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%.

In a further particular embodiment of the present invention, a high-purity febuxostat is provided, wherein the content of febuxostat is not less than 99.6%, and the content of 2-[3-carbamyl-4-(2-methylpropoxy)phenyl]-4-methyl-5-thiazole acid (FBZA) as impurity therein is not more than 0.4%, 0.3%, 0.2%, 0.15%, 0.1% or 0.05%, and the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%.

In a yet further particular embodiment of the present invention, a high-purity febuxostat is provided, wherein the content of febuxostat is not less than 99.7%, and the content of 2-[3-carbamyl-4-(2-methylpropoxy)phenyl]-4-methyl-5-thiazole acid (FBZA) as impurity therein is not more than 0.3%, 0.2%, 0.15%, 0.1% or 0.05%, and the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%.

In a yet further particular embodiment of the present invention, a high-purity febuxostat is provided, wherein the content of febuxostat is not less than 99.8%, and the content of 2-[3-carbamyl-4-(2-methylpropoxy)phenyl]-4-methyl-5-thiazole acid (FBZA) as impurity therein is not more than 0.2%, 0.15%, 0.1% or 0.05%, and the content of 2-[3-carboxy-4-(2-methylpropoxy)phenyl]-4-methyl-5-thiazole acid (FBZB) as impurity therein is not more than 0.2%, 0.15%, 0.1% or 0.05%.

In a yet further particular embodiment of the present invention, the content of febuxostat is not less than 99.9%, and the content of 2-[3-carbamyl-4-(2-methylpropoxy)phenyl]-4-methyl-5-thiazole acid (FBZA) as impurity therein is not more than 0.1% or 0.05%, and the content of 2-[3-carboxy-4-(2-methylpropoxy)phenyl]-4-methyl-5-thiazole acid (FBZB) as impurity therein is not more than 0.1% or 0.05%.

The present invention further provides a high-purity febuxostat, wherein the content of febuxostat is not less than 99.0%, and the content of 2-[3-carbamyl-4-(2-methylpropoxy)phenyl]-4-methyl-5-thiazole acid (FBZA) as impurity therein is not more than 0.5%, 0.3%, 0.2%, 0.15%, 0.1% or 0.05%, the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%, and the content of 2-[3-formyl-4-(2-methylpropoxy)phenyl]-4-methyl-5-thiazole acid (FBZC) as impurity therein is not more than 0.25%, 0.2%, 0.15%, 0.1% or 0.05%.

In another particular embodiment of the present invention, a high-purity febuxostat is provided, wherein the content of febuxostat is not less than 99.5%, and the content of 2-[3-carbamyl-4-(2-methylpropoxy)phenyl]-4-methyl-5-thiazole acid (FBZA) as impurity therein is not more than 0.5%, 0.4%, 0.3%, 0.2%, 0.15%, 0.1% or 0.05%, the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%, and the content of 2-[3-formyl-4-(2-methylpropoxy)phenyl]-4-methyl-5-thiazole acid (FBZC) as impurity therein is not more than 0.25%, 0.2%, 0.15%, 0.1% or 0.05%.

In another particular embodiment of the present invention, a high-purity febuxostat is provided, wherein the content of febuxostat is not less than 99.6%, and the content of 2-[3-carbamyl-4-(2-methylpropoxy)phenyl]-4-methyl-5-thiazole acid (FBZA) as impurity therein is not more than 0.4%, 0.3%, 0.2%, 0.15%, 0.1% or 0.05%, the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%, and the content of 2-[3-formyl-4-(2-methylpropoxy)phenyl]-4-methyl-5-thiazole acid (FBZC) as impurity therein is not more than 0.25%, 0.2%, 0.15%, 0.1% or 0.05%.

In another particular embodiment of the present invention, a high-purity febuxostat is provided, wherein the content of febuxostat is not less than 99.7%, and the content of the impurity 2-[3-carbamyl-4-(2-methylpropoxy)phenyl]-4-methyl-5-thiazole acid (FBZA) contained therein is not more than 0.3%, 0.2%, 0.15%, 0.1% or 0.05%, the content of 2-[3-carboxy-4-(2-methylpropoxy)phenyl]-4-methyl-5-thiazole acid (FBZB) as impurity therein is not more than 0.25%, 0.2%, 0.15%, 0.1% or 0.05%, and the content of 2-[3-formyl-4-(2-methylpropoxy)phenyl]-4-methyl-5-thiazole acid (FBZC) as impurity therein is not more than 0.25%, 0.2%, 0.15%, 0.1% or 0.05%.

In another particular embodiment of the present invention, a high-purity febuxostat is provided, wherein the content of febuxostat is not less than 99.8%, and the content of 2-[3-carbamyl-4-(2-methylpropoxy)phenyl]-4-methyl-5-thiazole acid (FBZA) as impurity therein is not more than 0.2%, 0.15%, 0.1% or 0.05%, the content of 2-[3-carboxy-4-(2-methylpropoxy)phenyl]-4-methyl-5-thiazole acid (FBZB) as impurity therein is not more than 0.2%, 0.15%, 0.1% or 0.05%, and the content of 2-[3-formyl-4-(2-methylpropoxy)phenyl]-4-methyl-5-thiazole acid (FBZC) as impurity therein is not more than 0.2%, 0.15%, 0.1% or 0.05%.

In another particular embodiment of the present invention, a high-purity febuxostat is provided, wherein the content of febuxostat is not less than 99.9%, and the content of 2-[3-carbamyl-4-(2-methylpropoxy)phenyl]-4-methyl-5-thiazole acid (FBZA) as impurity therein is not more than 0.1% or 0.05%, the content of 2-[3-carboxy-4-(2-methylpropoxy)phenyl]-4-methyl-5-thiazole acid (FBZB) as impurity therein is not more than 0.1% or 0.05%, and the content of 2-[3-formyl-4-(2-methylpropoxy)phenyl]-4-methyl-5-thiazole acid (FBZC) as impurity therein is not more than 0.1% or 0.05%.

Another purpose of the present invention is to provide a method for preparing the aforementioned high-purity febuxostat.

For this purpose, the present invention provides a method for preparing a high-purity febuxostat described as follows, comprising recrystallizing febuxostat in a mixed solvent comprising two or more solvents.

Specifically, the method can comprise:
(1) dissolving 2-[3-cyano-4-(2-methylpropoxy)phenyl)-4-methyl-5-thiazole carboxylic acid (febuxostat) in a mixed solvents under heating;
(2) cooling for crystallization;
(3) separating the crystallized 2-[3-cyano-4-(2-methylpropoxy)phenyl)-4-methyl-5-thiazole carboxylic acid (febuxostat) by filtration or centrifugation; and
(4) optionally, repeating the above steps according to the requirement of purity; and
(5) optionally, drying the separated product.

The mixed solvent used in the above purification method preferably comprises at least two types of alcohols, heterocyclic cycles, ketones, esters, ethers, and halogenated alkanes, wherein alcohols, ketones, esters, and heterocyclic cycles are preferred. On this basis, the mixed solvent can further comprise some other types of solvents, such as hydrocarbons, water, amides, sulfones, etc.

Preferably, the alcohol solvents aforementioned include methanol, ethanol, propanol, isopropanol, n-butanol, isobutanol, etc.; the heterocyclic cycle solvents include tetrahydrofuran, dioxane and thiazole; the ester solvents are ethyl acetate, methyl acetate, etc.; and the ketone solvents are acetone, cyclohexanone, and the like. The ratio of alcohols to heterocyclic cycles in the mixed solvent is generally 10:1 to 1:10 (volume/volume), more preferably 10:2 to 10:5 (volume/volume).

In order to obtain a higher yield of purification, it usually is preferred to use a relatively small amount of solvent and dissolve febuxostat at a relatively high temperature. The amount of solvent is usually from the amount of just dissolution to the amount of about 1-fold in excess, preferably from the amount of just dissolution to the amount of about 0.5-fold in excess. The dissolution temperature is generally from half of the boiling point of solvent to the boiling point of solvent, preferably the boiling point.

In step (1) of the above purification method, after febuxostat is dissolved completely, heat filtration may be carried out to remove some insoluble impurities therein.

In step (2) of the above purification method, the cooling for crystallization can be crystallization under stirring, or crystallization under standing. The final temperature for crystallization is generally from −20° C. to room temperature, preferably from −5° C. to room temperature.

In step (3) of the above purification method, a certain amount of a suitable solvent (e.g., solvents for crystallization or one of them) is generally used to wash the separated solid.

In step (5) of the above purification method, the drying is generally performed at a temperature of 50 to 150° C., preferably 70 to 90° C.; a vacuum generally of 0 to 0.098 MPa, preferably 0.08 to 0.095 MPa; and a drying time generally of 2 to 24 hours, preferably 8 to 16 hours.

The purification method can effectively reduce or remove impurities from a final product febuxostat. For example, when a mixed solvent of alcohol/heterocyclic cycle is used to purify a febuxostat, the febuxostat prior to purification has a FBZA content of about 1%, a FBZB content of about 0.5%, and a FBZC content of about 0.5%, while in the febuxostat product obtained by recrystallization once using a mixed solvent of methanol/tetrahydrofuran (volume ratio 5/1), the contents of FBZA, FBZB, and FBZC are less than 0.2%, 0.1% and 0.1%, respectively, and the content of other impurities is not more than 0.1%. If febuxostat is purified according to the methods described in literatures (such as WO9209279), recrystallization has to be repeated form many times to obtain a febuxostat with a relatively high purity, which will result in a lower yield of product and a significant increase of industrial cost. Therefore, the method for purifying febuxostat provided by the present invention is a new method for simple-to-operate, efficient and easy-to-industrialize purification of febuxostat.

The present invention further provides methods for preparing the above three impurity compounds: FBZA, FBZB and FBZC. By the methods, the three impurity compounds can be obtained in a purity of not less than 90%, preferably not less than 95%.

The method for preparing of 2-[3-carbamyl-4-(2-methylpropoxy)phenyl]-4-methyl-5-thiazole acid (FBZA) is as follows: hydrolyzing ethyl ester or methyl ester of 2-[3-cyano-4-(2-methylpropoxy)phenyl]-4-methyl-5-thiazole carboxylate (which can be prepared according to the method of JP10-45733) in a mixed solution comprising an alkali solution and an organic solvent, neutralizing with an acid after the end of hydrolysis, separating and drying. The obtained product can be further purified via recrystallization. In the method, the alkali solution is preferably an aqueous solution of sodium hydroxide or potassium hydroxide, having a concentration of 0.5-5.0 mol/L, preferably of 2-3 mol/L; the organic solvent is preferably selected from tetrahydrofuran, methanol, ethanol and acetone; the reaction temperature is preferably 70-80° C.; the hydrolysis time is 18 to 36 hours, preferably 20 to 30 hours; the acid for neutralization is preferably hydrochloric acid; the solvent for recrystallization can be methanol, ethanol, etc.; the drying temperature is generally 50-150° C., preferably 70-90° C.; the vacuum is usually 0-0.098 MPa, preferably 0.08-0.095 MPa.

The method for preparing 2-[3-carboxy-4-(2-methylpropoxy)phenyl]-4-methyl-5-thiazole acid (FBZB) is as follows: hydrolyzing ethyl ester or methyl ester of 2-[3-cyano-4-(2-methylpropoxy)phenyl]-4-methyl-5-thiazole carboxylate (which can be prepared according to the method of JP10-45733) in a mixed solution comprising an alkali solution and an organic solvent, neutralizing with an acid after the end of hydrolysis, separating and drying. The obtained product can be further purified via recrystallization. In the method, the alkali solution is preferably an aqueous solution of sodium hydroxide or potassium hydroxide, having a concentration of 0.5-5.0 mol/L, preferably of 2-3 mol/L; the organic solvent is preferably selected from tetrahydrofuran, methanol, ethanol and acetone; the reaction temperature is preferably 80-90° C.; the hydrolysis time is 36 to 72 hours, preferably 44 to 52 hours; the acid for neutralization is preferably hydrochloric acid; the solvent for recrystallization can be selected from methanol, ethanol, etc.; the drying temperature is generally 50-150° C., preferably 70-90° C.; the vacuum is usually 0-0.098 MPa, preferably 0.08-0.095 MPa.

The method for preparing 2-[3-formyl-4-(2-methylpropoxy)phenyl]-4-methyl-5-thiazole acid (FBZC) is as follows: hydrolyzing ethyl ester or methyl ester of 2-[3-cyano-4-(2-methylpropoxy)phenyl]-4-methyl-5-thiazole carboxylate (which can be prepared according to the method of JP10-45733) in a mixed solution comprising an alkali solution and an organic solvent, neutralizing with an acid after the end of hydrolysis, separating and drying. The obtained product can be further purified via recrystallization. In the method, the alkali solution is preferably an aqueous solution of sodium hydroxide or potassium hydroxide, having a concentration of 0.5-5.0 mol/L, preferably of 2-3 mol/L; the organic solvent is preferably selected from tetrahydrofuran, methanol, ethanol and acetone; the reaction temperature is preferably 60-90° C.; the hydrolysis time is 0.5 to 20 hours, preferably 1 to 6 hours; the acid for neutralization is preferably hydrochloric acid; the solvent for recrystallization can be selected from ethyl acetate, methanol, ethanol, etc.; the drying temperature is generally 50-150° C., preferably 70-90° C.; the vacuum is usually 0-0.098 MPa, preferably 0.08-0.095 MPa.

In the present invention, the contents of febuxostat and impurities therein are determined by high performance liquid chromatography (HPLC, detection wavelength of about 220 nm) using area normalization method, which has a detection limit of not lower than 0.02% and a quantification limit of not lower than 0.05%. The purities of FBZA, FBZB and FBZC are also determined by high performance liquid chromatography (HPLC, detection wavelength of about 220 nm) using area normalization method. The values of contents or purities are obtained by rounding the tested data.

Accordingly, the present invention further provides a use of the above impurities as standard references for quality control of the high-purity febuxostat of the present invention. In one embodiment, the present invention provides a use of the impurities FBZA and/or FBZB as standard references for quality control of the high-purity febuxostat of the present invention. In another embodiment, the present invention provides a use of the impurities FBZA and/or FBZB and/or FBZC as standard references for quality control of the high-purity febuxostat of the present invention.

According to another aspect, the present invention provides a pharmaceutical composition comprising the above high-purity febuxosta and optional pharmaceutically acceptable adjuvants including excipients and carriers. The pharmaceutical composition can be in the form of pharmaceutical preparation, such as tablets, capsules and other solid or dry pharmaceutical preparations. Among them, tablet form is preferred. These preparations can be made by those skilled in the art using corresponding known adjuvants according to common pharmaceutical preparation technologies known. For example, a tablet can contain a filler, a binder, a disintegrant, a lubricant and other excipients; the filler can be one or more selected from the group consisting of microcrystalline cellulose, lactose, pregelatinized starch, mannitol, starch and sorbitol, preferably lactose and microcrystalline cellulose; the disintegrant can be one or more selected from the group consisting of cross-linked sodium carboxymethyl cellulose, low substituted hydroxypropyl cellulose, sodium carboxymethyl starch and cross-linked polyvidone, preferably cross-linked sodium carboxymethyl cellulose; the binder can be one or more selected from the group consisting of polyvidone, starch slurry and hydroxypropyl methyl cellulose, preferably hydroxypropyl methylcellulose cellulose; the lubricant can be magnesium stearate and the like. The febuxostat tablet described in the present invention is prepared by wet granulation and tableting technology, comprising pulverization, sieving, mixing, granulation, drying, sizing, tableting and the like.

The present invention further provides a use of the above high-purity febuxostat or the pharmaceutical composition comprising the above high-purity febuxostat in the manufacture of a medicament for prophylaxis or treatment of a disease associated with high level of blood uric acid, the disease associated with high level of blood uric acid mainly refers to a gout caused by high level of blood uric acid, a condition of high level of blood uric acid caused by chemotherapy in cancer patients and other diseases associated with high level of blood uric acid.

The present invention also provides a method for prophylaxis or treatment of a disease, including gout, associated with high level of uric acid, comprising administering a patient in need of the treatment with the high-purity febuxostat of the present invention.

### EXAMPLES

The following examples are used to illustrate the present invention, but not intended to limit the scope of the present invention.

### Example 1

### Preparation of 2-[3-carbamoyl-4-(2-methylpropoxy)phenyl]-4-methyl-5-thiazole carboxylic acid (FBZA)

To a 500 ml three-necked bottle was added 20.0 g of ethyl 2-[3-cyano-4-(2-methyl propoxy)phenyl]-4-methyl-5-thiazolcarboxylate, 200 ml of 10% sodium hydroxide solution and 70 ml of tetrahydrofuran, the reaction was performed under stirring at 75-80° C. for about 24 h, cooled, then adjusted using a concentrated hydrochloride to about pH 3. A white solid was precipitated and filtered, and the filter cake was washed with water and then recrystallized with methanol. The precipitate was collected by filtration and the filter cake was dried at 80-85° C. in a reduced pressure (−0.085 to −0.090 MPa) to obtain 2-[3-carbamoyl-4-(2-methylpropoxy)phenyl]-4-methyl-5-thiazolcarboxylic acid (FBZA) (9.4 g) as a white crystalline. Purity (by HPLC): 98%. IR (KBr): 3464, 3399, 3190, 2963, 1693, 1646, 1598, 1505, 1411, 1256, 1161, 1016 cm⁻¹. ¹H-NMR (500 MHz, DMSO-d₆) δ (ppm): 8.342-8.346 (1H, d), 8.013-8.035 (1H, m), 7.255-7.273 (1H, d), 3.972-3.985 (2H, d), 2.095-2.148 (1H, s), 2.656 (3H, m), 0.997-1.010 (6H, d).

### Example 2

### Preparation of 2-[3-carboxyl-4-(2-methylpropoxy)phenyl]-4-methyl-5-thiazole carboxylic acid (FBZB)

To a 500 ml three-necked bottle was added 22.0 g of ethyl 2-[3-cyano-4-(2-methyl propoxy)phenyl]-4-methyl-5-thiazolcarboxylate, 220 ml of 10% sodium hydroxide solution and 80 ml of tetrahydrofuran, the reaction was performed by heating to reach intense reflux for about 48 h, then the heating stopped, cooled, and adjusted by adding dropwise slowly a concentrated hydrochloride to about pH 3. A solid was precipitated and vacuum filtered. The filter cake was recrystallized with absolute ethanol, filtered under vacuum, then dried at 75-85° C. at a reduced pressure (−0.090 to −0.095 MPa) to obtain 2-[3-carboxy-4-(2-methylpropoxy)phenyl]-4-methyl-5-thiazole acid (FBZB) (8.7 g) as a white-off solid. Purity (by HPLC): 98%. IR (KBr): 3396, 2959, 2874, 1692, 1604, 1508, 1422, 1377, 1293, 1252, 1223, 1167, 1111, 1092, 1017, 825 cm⁻¹. ¹H-NMR (500 MHz, DMSO-d₆) δ (ppm): 8.185-8.181 (1H, d), 7.958-7.938 (1H, m), 7.125-7.107 (1H, d), 3.825-3.812 (2H, d), 2.614 (3H, s), 2.031-1.979 (1H, m), 0.971-0.958 (6H, d).

### Example 3

### Preparation of 2-[3-formyl-4-(2-methylpropoxy)phenyl]-4-methyl-5-thiazole carboxylic acid (FBZC)

To a 500 ml three-necked bottle was added 28.0 g of ethyl 2-[3-formyl-4-(2-methyl propoxy)phenyl]-4-methyl-5-thiazolcarboxylate, 280 ml of 10% sodium hydroxide solution and 90 ml of ethanol, the reaction was performed under stirring at about 80° C. for about 4 h, then the reaction stopped, cooled, adjusted by adding dropwise slowly a concentrated hydrochloride to about pH 3. A white solid was precipitated and filtered. The filter cake was washed with water, and dried by vacuum sucking. The filter cake was recrystallized with ethyl acetate, filtered, then dried at 70-75° C. in a reduced pressure (−0.080 to −0.085 MPa) to obtain 2-[3-formyl-4-(2-methylpropoxy)phenyl]-4-methyl-5-thiazole carboxylic acid (FBZC) (11.2 g) as a white crystalline. Purity (by HPLC): 99%. IR (KBr): 3432, 2966, 2871, 1679, 1652, 1605, 1513, 1447, 1427, 1371, 1179, 1111, 1014 cm⁻¹. ¹H-NMR (500 MHz, DMSO-d₆) δ (ppm): 13.360 (1H, s), 10.397 (1H, s), 8.191-8.153 (2H, m), 7.337-7.319 (1H, d), 3.990-3.977 (2H, d), 2.659 (3H, s), 2.163-2.084 (1H, s), 1.045-1.031 (6H, d).

### Example 4

### Preparation of High-Purity Febuxostat

To a 1 L reaction flask was added 70 g of crude febuxostat (HPLC: 98.1%, sic passim), 480 ml of methanol and 95 ml of tetrahydrofuran, heated under refluxing to dissolve febuxostat, filtered while hot, and the filtrate was allowed to stand for crystallization. A white crystal was precipitated and filtered. The filter cake was vacuum dried (80-85° C., −0.080 to −0.090 MPa) for more than 8 h to obtain a white crystal (59.6 g). HPLC: 99.9%, FBZA: 0.02%, FBZB: 0.03%, FBZC: 0.03%.

### Example 5

### Preparation of High-Purity Febuxostat

To a 200 ml reaction flask was added 10 g of crude febuxostat, 60 ml of methanol and 24 ml of tetrahydrofuran, heated under refluxing to dissolve febuxostat, filtered while hot. The filtrate was stirred for crystallization, and then filtered. The filter cake was vacuum dried (75-80° C., −0.090 to −0.095 MPa) for more than 15 h to obtain a white crystalline (7.91 g). HPLC: 99.9%, FBZA: 0.02%, FBZB: 0.01%, FBZC: 0.02%.

### Example 6

### Preparation of High-Purity Febuxostat

To a 200 ml reaction flask was added 10 g of crude febuxostat, 80 ml of ethanol and 20 ml of tetrahydrofuran, heated under refluxing to dissolve febuxostat, cooled the filtrate to 0-5° C., and filtered. The filter cake was dried in vacuum (70-75° C., −0.08 to −0.09 MPa) for more than 8 h to obtain a white crystalline (7.54 g). HPLC: 99.8%, FBZA: 0.04%, FBZB: 0.06%, FBZC: 0.06%.

### Example 7

### Preparation of High-Purity Febuxostat

To a 100 ml reaction flask was added 10 g of crude febuxostat, 50 ml of acetone and 15 ml of tetrahydrofuran, heated under refluxing to dissolve febuxostat, filtered while hot, and the filtrate was stirred and cooled to 5-10° C., then filtered. The filter cake was washed with a small amount of acetone, then dried in vacuum (85-90° C., −0.085 to −0.095 MPa) for more than 9 h to obtain a white crystalline (7.25 g). HPLC: 99.6%, FBZA: 0.15%, FBZB: 0.08%, FBZC: 0.12%.

### Example 8

### Preparation of High-Purity Febuxostat

To a 200 ml reaction flask was added 10 g of crude febuxostat, 50 ml of methanol, 10 ml of water and 24 ml of tetrahydrofuran, heated under refluxing to dissolve febuxostat, filtered while hot, and the filtrate was cooled and filtered. The filter cake was washed with a small amount of methanol, and then dried in vacuum (70-75° C., −0.080 to −0.085 MPa) for more than 16 h to obtain a white crystalline (8.11 g). HPLC: 99.5%, FBZA: 0.30%, FBZB: 0.11%, FBZC: 0.07%.

### Example 9

### Preparation of High-Purity Febuxostat

To a 200 ml reaction flask was added 10 g of crude febuxostat, 60 ml of acetone and 30 ml of ethyl acetate, heated under refluxing to dissolve febuxostat, filtered while hot, and the filtrate was cooled and filtered. The filter cake was dried in vacuum (80-85° C., −0.085 to −0.095 MPa) for more than 10 h to obtain a white crystalline (7.89 g). HPLC: 99.7%, FBZA: 0.12%, FBZB: 0.07%, FBZC: 0.10%.

### Example 10

### High-Purity Febuxostat Tablet

### Formula for Per Tablet

| | |
|---|---|
| Febuxostat (HPLC: >99.7%, FBZA < 0.1%, FBZB < | 80 mg |
| 0.1%, FBZC < 0.1%) | |
| Lactose | 160 mg |
| microcrystalline cellulose | 40 mg |
| cross-linked sodium carboxymethyl cellulose | 10 mg (70% for |
| | inner addition) |
| magnesium stearate | 3 mg |
| 1% aqueous solution of hydroxypropyl methyl | q.s. |
| cellulose | |

### Preparation Process:

1. Febuxostat was micronized to have a particle diameter of 0-10 μm, ready for use;
2. Lactose, microcrystalline cellulose, cross-linked sodium carboxymethyl cellulose and magnesium stearate were passed through a sieve of 80 mesh, ready for use;
3. 1% aqueous solution of hydroxypropyl methyl cellulose was prepared with purified water, ready for use;
4. Febuxostat, lactose, microcrystalline cellulose, and cross-linked sodium carboxymethyl cellulose for inner addition were mixed homogenously, added with a binder to make dough, and the dough was passed through a sieve of 20 mesh to prepare wet granules. The wet granules were dried at 50° C.-60° C., and the dried granules were passed through sieve for sizing. Then magnesium stearate and cross-linked sodium carboxymethyl cellulose for outer addition were added and mixed homogenously to obtain a semi-finished product. Assay was performed and tablet weight was calculated. The finished produced was obtained by tableting in which tablet weight was adjusted according to the content of the semi-finished product.

1. A high-purity febuxostat, characterized by having a febuxostat content of not less than 99.0%.

2. The high-purity febuxostat of claim 1, which is obtained by recrystallizing a febuxostat in a mixed solvent comprising two or more solvents.

3. The high-purity febuxostat of claim 2, wherein the solvents are selected from alcohols, heterocyclic cycles, ketones, esters, ethers and halogenated alkanes.

4. The high-purity febuxostat of claim 3, wherein the solvents are selected from alcohols, ketones, esters or heterocyclic cycles.

5. The high-purity febuxostat of claim 2, wherein the alcohol solvents are selected from methanol, ethanol, propanol, isopropanol, n-butanol and isobutanol; the ketone solvents are selected from acetone and cyclohexanone; the ester solvents are selected from ethyl acetate and methyl acetate; the heterocyclic cycle solvents are selected from tetrahydrofuran, dioxane and thiazole.

6. The high-purity febuxostat of claim 3, wherein the solvent is a mixed solvent of an alcohol and a heterocyclic cycle in a volume ratio of 10:1 to 1:10, preferably 10:2 to 10:5.

7. The high-purity febuxostat of claim 6, wherein the solvent is a mixed solvent of methanol and tetrahydrofuran in a volume ratio of 5:1.

8. The high-purity febuxostat of claim 1, wherein the content of 2-[3-carbamyl-4-(2-methylpropoxy)phenyl]-4-methyl-5-thiazole acid (FBZA) as impurity contained therein is not more than 0.5%, and the content of 2-[3-carboxy-4-(2-methylpropoxy)phenyl]-4-methyl-5-thiazole acid (FBZB) as impurity contained therein is not more than 0.25%.

9. The high-purity febuxostat of claim 1, wherein the content of 2-[3-carbamyl-4-(2-methylpropoxy)phenyl]-4-methyl-5-thiazole acid (FBZA) as impurity contained therein is not more than 0.5%, the content of 2-[3-carboxy-4-(2-methylpropoxy)phenyl]-4-methyl-5-thiazole acid (FBZB) as impurity contained therein is not more than 0.25%, and the content of 2-[3-formyl-4-(2-methylpropoxy)phenyl]-4-methyl-5-thiazole acid (FBZC) as impurity contained therein is not more than 0.25%.

10. The high-purity febuxostat of claim 8, wherein the content of FBZA as impurity contained therein is not more than 0.2%, and the content of FBZB as impurity contained therein is not more than 0.1%.

11. The high-purity febuxostat of 9, wherein the content of FBZC as impurity contained therein is not more than 0.1%.

12. A method for preparing the high-purity febuxostat of claim 1, the method comprising recrystallizing a febuxostat in a mixed solvent comprising two or more solvents.

13. The preparation method of claim 12, wherein the solvents are selected from alcohols, heterocyclic cycles, ketones, esters, ethers, halogenated hydrocarbons, especially alcohols, ketones, esters or heterocyclic cycles.

14. The preparation method of claim 13, wherein the solvent is a mixed solvent of an alcohol and a heterocyclic cycle in a volume ratio of 10:1 to 1:10, preferably 10:2 to 10:5.

15. The preparation method of claim 13, wherein the alcohol solvents are selected from methanol, ethanol, propanol, isopropanol, n-butanol and isobutanol; the ketone solvents are selected from acetone, cyclohexanone; the ester solvents are selected from ethyl acetate and methyl acetate; and the heterocyclic cycle solvents are selected from tetrahydrofuran, dioxane and thiazole.

16. A method for using of 2-[3-carbamyl-4-(2-methylpropoxy)phenyl]-4-methyl-5-thiazole acid (FBZA) and/or 2-[3-carboxyl-4-(2-methylpropoxy)phenyl]-4-methyl-5-thiazole acid (FBZB) as standard references for quality control of the high-purity febuxostat of claim 1.

17. A method for using of 2-[3-carbamyl-4-(2-methylpropoxy)phenyl]-4-methyl-5-thiazole acid (FBZA) and/or 2-[3-carboxyl-4-(2-methylpropoxy)phenyl]-4-methyl-5-thiazole acid (FBZB) and/or 2-[3-formyl-4-(2-methylpropoxy)phenyl]-4-methyl-5-thiazole acid (FBZC) as standard references for quality control of the high-purity febuxostat of claim 1.

18.-20. (canceled)

21. 2-[3-carbamyl-4-(2-methylpropoxy)phenyl]-4-methyl-5-thiazole acid.

22. 2-[3-carboxy-4-(2-methylpropoxy)phenyl]-4-methyl-5-thiazole acid.
